(19) United States
(12) Patent Application Publication
(10) Pub. No.: US 2017/0119783 A1
(43) Pub. Date: May 4, 2017

(54) Method for Treating Cognitive Impairment Associated With Neurodegenerative Disorders

(71) Applicant: Peter Parsonson, Austin, TX (US)

(72) Inventor: Peter Parsonson, Austin, TX (US)

(21) Appl. No.: 15/238,704

(22) Filed: Aug. 16, 2016

Related U.S. Application Data
(63) Continuation-in-part of application No. 14/585,876, filed on Dec. 30, 2014, now abandoned.

(51) Int. Cl. A61K 31/541

(52) U.S. Cl. CPC A61K 31/541

(57) ABSTRACT

A method for treating Alzheimer's Disease (AD) by administering an efficacious amount of artemisone. It is hypothesized that the treatment counters AD by inhibiting the production of the amyloid protein which has been implicated as a contributing factor to the onset of AD and the associated AD dementia and memory loss. Additionally, artemisone acts to reduce inflammation in the brain associated with AD by inhibiting tumor necrosis factor (TNF). Artemisone is a particularly useful AD treatment as it is able to cross the blood-brain barrier whereas many other TNF inhibitors are unable to do so. Artemisone may be administered via the following routes: subcutaneous, intravenous, intrathecal, intramuscular, intranasal, oral, transepidermal, parenteral, by inhalation, or intracerebroventricular.

### BACKGROUND OF THE INVENTION

Alzheimer's disease is a disease characterized by degeneration and loss of neurons and also by the formation of senile plaques and neurofibrillary change. Presently, treatment of Alzheimer's disease is limited to symptomatic therapies only. A basic remedy which prevents progress of the disease has not been developed. A method of controlling the cause of onset of pathologic conditions needs to be developed for creation of the basic remedy of Alzheimer's disease.

A.beta. protein, which is a metabolite of amyloid precursor protein (hereinafter referred to as APP), is considered to be greatly involved in degeneration and loss of neurons as well as onset of dementia related conditions (for example, see Klein W L, et al Proceeding National Academy of Science USA, Sep. 2, 2003, 100(18), p. 10417-22, suggest a molecular basis for reversible memory loss.

Kitsch R M, and 16 others, Antibodies against Beta-amyloid slow cognitive decline in Alzheimer's disease, Neuron, May 22, 2003, 38(4), p. 547-554) suggest that the main components of A.beta. protein are A.beta.40 consisting of 40 amino acids and A.beta.42 having two additional amino acids at the C-terminal. The A.beta.40 and A.beta.42 tend to aggregate (for example, see Jarrell J T et al, The carboxy terminus of the .beta. amyloid protein is critical for the seeding of amyloid formation: implications for the pathogenesis of Alzheimer's disease, Biochemistry, May 11, 1993, 32(18), p. 4693-4697) and constitute main components of senile plaques (for example, (Glenner G G, et al, Alzheimer's disease: initial report of the purification and characterization of a novel cerebrovascular amyloid protein, Biochemical and Biophysical Research Communications, May 16, 1984, 120(3), p. 885-90. See also Masters C L, et al, Amyloid plaque core protein in Alzheimer disease and Down syndrome, Proceeding National Academy of Science USA, June 1985, 82(12), p. 4245-4249.).

Furthermore, it is known that mutations of APP and presenelin genes, which are observed in familial Alzheimer's disease, increase the production of A.beta.40 and A.beta.42 (for example, see Gouras G K, et al, Intraneuronal A.beta.42 accumulation in human brain, American Journal of Pathology, January 2000, 156(1), p. 15-20. Also, see Scheuner D, et al, Nature Medicine, August 1996, 2(8), p. 864-870; and Forman M S, et al, Differential effects of the Swedish mutant amyloid precursor protein on .beta.-amyloid accumulation and secretion in neurons and normeuronal cells, Journal of Biological Chemistry, Dec. 19, 1997, 272(51), p. 32247-32253.). Therefore, compounds which reduce production of A.beta.40 and A.beta.42 are expected as an agent for controlling progress of Alzheimer's disease or for preventing the disease entirely.

Also of interest in connection with the present invention are: US 2007/0117798 (Eisai, published May 24, 2007); US 2007/0117839 (Eisai, published May 24, 2007); US 2006/0004013 (Eisai, published Jan. 5, 2006); WO 2005/110422 (Boehringer Ingelheim, published Nov. 24, 2005); WO 2006/045554 (Cellzone AG, published May 4, 2006); WO 2004/110350 (Neurogenetics™, published Dec. 23, 2004); WO 2004/071431 (Myriad Genetics, published Aug. 26, 2004); US 2005/0042284 (Myriad Genetics, published Feb. 23, 2005) and WO 2006/001877 (Myriad Genetics, published Jan. 5, 2006).

There is a need for new compounds, formulations, treatments and therapies to treat diseases and disorders associated with A.beta accumulation. It is, therefore, an object of this invention to provide compounds useful in the treatment or prevention or amelioration of such diseases and disorders.

### FIELD OF THE INVENTION

The present invention relates to a certain sesquiterpene lactone compound, artemisone, that has proven to be useful as an A.beta inhibitor as well as pharmaceutical compositions containing the compound, and methods of treatment using the compound to treat various diseases including central nervous system disorders such as, for example, neurodegenerative diseases such as Alzheimer's disease and other diseases relating to the deposition of amyloid protein. This compound is especially useful for reducing Amyloid beta (hereinafter referred to as A.beta. production which is effective in the treatment of diseases caused by A.beta. such as, for example, Alzheimers Disease.

### BRIEF DESCRIPTION OF THE DRAWINGS

FIG. 1 shows the chemical structure of Artemisone and its endoperoxide bridge.

### DETAILED DESCRIPTION AND BEST MODE OF IMPLEMENTATION

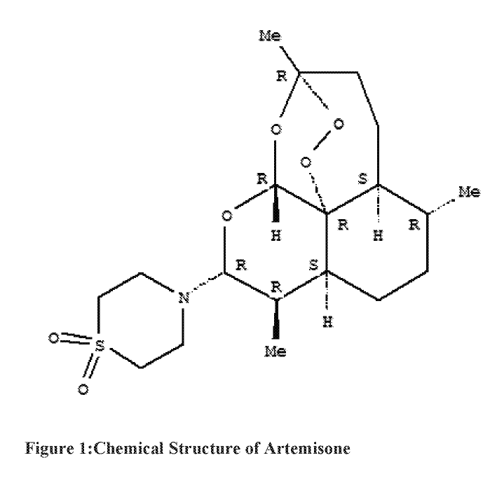

This invention relates to a certain extract of the Artemisia annua plant, both in their crude and refined forms, and certain refined forms of Artemisia annua plant extracts composed substantially of Artemisone FIG. 1, which is chemically classified as a sesquiterpene lactone compound containing an endo-peroxide bridge. The compound is suitable for subcutaneous, intravenous, intrathecal, intramuscular, intranasal, oral, transepidermal, parenteral, by inhalation, or intracerebroventricular application for the treatment of Alzheimer's Disease and its related dementia.

The present invention discloses a method of topical, parenteral, or oral administration of Artemisone as a refined form from the Artemisia annua plant composed substantially of Artemisone. This method can include a base, a carrier, a solvent or a delivery system.

Artemisia annua extracts have been used in ancient Chinese medicine (qinqhao) for a number of treatments. Medicinal use of the Chinese herb qinqhao appears in several standard Chinese Materia Medica texts as a treatment for febrile illnesses. The herb was specifically recommended for fevers in the Zhou Hou Bei fi Fang (The Handbook of Prescriptions for Emergencies) written by Ge Heng and published in 341 AD. The most detailed description appears in the “Compendium of Materia Medica—Ben Cao Gang Mu, compiled in 1596, and is still printed in China today. The antimalarial activity of qinqhao was rediscovered in China in 1972, and the antimalarial active principal of qinqhao was named “qinghaosu”. The western name for the compound is artemisinin. Recently, however, this extract and its sesquiterpene derivatives have been used for the treatment of malaria. Distribution of artemisinin in Artemisia annua has been reviewed [Ferreira et al., Progress in New Crops, J. Janick (ed.), ASHS Press 579 (1996)].

Artemisinin (Qinghaosu) and its analogs including Artemisone are the treatments of choice for cerebral or chloroquine resistant malaria or for patients with chloroquine allergy. Artemisone is a sesquiterpene lactone with a peroxide bridge, and is characterized by very low toxicity. Artemisone stimulates cell-mediated immunity, and yet decreases abnormally elevated levels of polyamine regulatory proteins. It also markedly inhibits nucleic acid and protein syntheses. Further, it affects cellular membrane functions and decreases hepatic cytochrome oxidase enzyme system activity. Still further, it is virustatic against influenza and cidal against three groups of pathogenic parasites.

The very low toxicity of this compound to humans is a major benefit. For example, artesunate is twice as safe as artemether and only one-fiftieth as toxic as chloroquinine, the most common antimalarial. The first manifestation of toxicity of these compounds is generally a decreased reticulocyte count. Other manifestations include transient fever, decreased appetite and elevated blood transaminase levels, the latter an indication of hepatotoxicity.

In the present invention it is thus both surprising and unexpected that Artemisone can be utilized in topical, parenteral and orally administered formulations as a treatment for Alzheimer's Disease.

### DESCRIPTION OF THE RELATED ART

Artemisia annua extracts have been used in ancient Chinese medicine (qinhao) for a number of treatments. Medicinal use of the Chinese herb qinqhao appears in several standard Chinese Medical texts as a treatment for febrile illnesses. The herb was specifically recommended for fevers in the Zhou Hou Bei fi Fang (The Handbook of Prescriptions for Emergencies) written by Ge Heng and published in 341 AD. The most detailed description appears in the “Compendium of Materia Medica—Ben Cao Gang Mu, compiled in 1596, and is still printed in China today. The antimalarial activity of qinqhao was rediscovered in China in 1972, and the antimalarial active principal of qinqhao was named “qinghaosu”. The western name for the compound is artemisinin. Recently, however, this extract and its sesquiterpene deriviatives have been used for the treatment of malaria. Distribution of artemisinin in Artemisia annua has been reviewed [Ferreira et al., Progress in New Crops, J. Janick (ed.), ASHS Press 579 (1996)].

Artemisinin (Qinghaosu) and its analogs are the treatments of choice for cerebral or chloroquine resistant malaria or for patients with chloroquine allergy, Artemisinin is a naturally occurring substance, obtained by purification from sweet wormwood, Artemisia annua. Artemisinin and its analogs are sesquiterpene lactones with a peroxide bridge, and are characterized by very low toxicity and poor water solubility. Artemisinin is known as a humoral immunosuppressive agent that is less active than cyclophosphamide, the latter being one of the major chemotherapeutic agents for carcinomas. Artemisinin stimulates cell-mediated immunity, and yet decreases abnormally elevated levels of polyamine regulatory proteins. It also markedly inhibits nucleic acid and protein syntheses. Further, it affects cellular membrane functions and decreases hepatic cytochrome oxidase enzyme system activity. Still further, it is virustatic against influenza and cidal against three groups of pathogenic parasites.

Known analogs of artemisinin that have higher solubility in water are dihydroartemisinin, artemether, artesunate, arteether, propylcarbonate dihydroartemisinin and artelinic acid. Dihydroartemisinin has an antimalarial potency that is 60% higher than that of artemisinin. Artemether and artesunate have antimalarial potencies that are 6 times and 5.2 times, respectively, that of artemisinin. In terms of their ability to inhibit nucleic acid synthesis, dihydroartemisinin, artemether, artesunate, arteether, and propylcarbonate dihydroartemisinin all have 100 times the activity of artemisinin, and protein synthesis is stimulated to an even greater extent by these compounds. Artesunate stimulates the immune system at low doses and inhibits it at high doses. Artelinic acid is the most water-soluble and the most stable of the group. Two of the compounds in this group have been demonstrated to display synergistic activity with doxorubicin (a chemotherapeutic agent) and miconazole (an antifungal agent) in the in vitro killing of Plasmodium falciparum, the etiologic agent of malaria. Anemisone has recently been disclosed to possess high antimalarial activity [Haynes et al., Angew. Chem. Int. Ed. Engl., 20, 2082 (2006)]. Secondary metabolites of Artemisia annua extract and their biological activity has been reviewed [Bhakuni et al., Current Science, 80, 35 (2001)].

The very low toxicity of these compounds to humans is a major benefit. Artesunate, for example, is twice as safe as artemether and only one-fiftieth as toxic as chloroquinine, the most common antimalarial. The first manifestation of toxicity of these compounds is generally a decreased reticulocyte count. Other manifestations include transient fever, decreased appetite and elevated blood transaminase levels, the latter an indication of hepatotoxicity.

U.S. Pat. No. 8,193,376 discloses the use of artemisinin and the active principles contained in Artemisia annua extracts in conjunction with amino acids, peptides, and amino sugars, and isomers and salts thereof for the treatment of parasitic infections.

U.S. Pat. No. 8,232,417 discloses the use of artemisinin and its endoperoxide derivatives for the treatment of infections caused by Propionibacterium acnes and Pityrosporum globosa as well as skin disorders such as skin wrinkles and fine lines, dandruff, and age spots.

U.S. Pat. No. 4,978,676 discloses the use of artemisinin and artemisinin analogs in the treatment of skin conditions such as psoriasis, blistering skin diseases, viral warts, and hemorrhoids.

U.S. Pat. No. 5,219,880 discloses the use of artemisinin and artemisinin analogs in the treatment of warts, molluscum contagiosum and hemorrhoids.

U.S. Pat. No. 5,225,427 discloses certain 10-substituted ether derivatives of dihydroartemisinin alleged to exhibit antimalarial and antiprotozoal activity.

Artemisinin [(3R,5aS,6R,8aS,9R,12S,12aR)-octahydro-3,6,9-trimethyl-3,12-epoxy-12H-pyr-ano[4,3-j]-1,2-benzodioxepin-10(3H)-one, formula (I)] alone has been shown to be toxic to cancer cells in vitro (Sun et al., “Antitumor Activities of 4 Derivatives of Artemisic Acid and Artemisinin B in vitro,” Chung-Kuo-Yao-Li-Hsuch-Pao 13:541-543 (1992)). The effect was found to be more effective for hepatoma and embryonic lung cells than against human gastric cancer cells. in another study (Woerdenbag et al., “Cytotoxicity of Artemisinin related Endoperoxides to Erich Ascots Tumor Cells,” J. Nat. Prod. 56(6): 849-856 (1993)), artemisinin was shown to have efficacy against Ehrlich ascots tumor cells, as were several derivatives of dihydroarteinisinin (artemether, arteether, sodium artesunate, artelinic acid, and sodium artelinate).

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

What is claimed is:

1. The use of Artemisone as a treatment for for cognitive impairment and dementia related to the accumulation of A.Beta. via a route of administration to include subcutaneous, intravenous, intrathecal, intramuscular, intranasal, oral, transepidermal, parenteral, by inhalation, or intracerebroventricular.

2. The method of claim 1, wherein the treatment is for the restoration of memory function.

3. The method of claim 1, wherein the treatment is for the restoration of performance of activities of daily living (ADL) capacity.
